(19) United States
(12) Patent Application Publication
(10) Pub. No.: US 2020/0022621 A1
(43) Pub. Date: Jan. 23, 2020

(54) Apparatus for a Shoe and Method

(71) Applicants: Kenneth Perlin, New York, NY (US); Charles Hendee, Manteca, CA (US); Michael Gold, New York, NY (US)

(72) Inventors: Kenneth Perlin, New York, NY (US); Charles Hendee, Manteca, CA (US); Michael Gold, New York, NY (US)

(73) Assignees: Tactonic Technologies, LLC, New York, NY (US); New York University, New York, NY (US)

(21) Appl. No.: 16/514,597

(22) Filed: Jul. 17, 2019

Related U.S. Application Data
(60) Provisional application No. 62/700,035, filed on Jul. 18, 2018.

(51) Int. Cl. A61B 5/11; A43B 3/00; A61B 5/00; A43B 13/16

(52) U.S. Cl. CPC A61B 5/11; A43B 13/16; A61B 5/6807; A43B 3/0005

(57) ABSTRACT

An apparatus for a shoe which provides information to a remote computing device having a user application. The apparatus includes a first insert configured to be disposed in the shoe having a wireless transmitter, at least one sensor for sensing pressure that provides an antialiased image of pressure, and a controller in communication with the sensor and the transmitter which receives the pressure sensed by the sensor and transmits data associated with the pressure sensed through the transmitter to the device. A method for providing information about a user disposed in a first shoe of the user. A wireless pressure sensitive shoe insert.

### CROSS-REFERENCE TO RELATED APPLICATIONS

This is a non-provisional application of U.S. provisional application Ser. No. 62/700,035 filed Jul. 18, 2018, incorporated by reference herein.

### FIELD OF THE INVENTION

The present invention is related to an apparatus for a shoe which has at least one sensor for sensing pressure that provides an antialiased image of pressure. (As used herein, references to the “present invention” or “invention” relate to exemplary embodiments and not necessarily to every embodiment encompassed by the appended claims.) More specifically, the present invention is related to an apparatus for a shoe which has an insert with at least one sensor for sensing pressure that provides an antialiased image of pressure, that can target any desired region or regions of the foot wearing the shoe to detect changes in pressure as well as to detect features such as phalanges, tarsals, metatarsals and the heel of the foot.

### BACKGROUND OF THE INVENTION

This section is intended to introduce the reader to various aspects of the art that may be related to various aspects of the present invention. The following discussion is intended to provide information to facilitate a better understanding of the present invention. Accordingly, it should be understood that statements in the following discussion are to be read in this light, and not as admissions of prior art.

Walking, or for that matter more generally moving legs, is one of the most basic movements of people. Ordinarily, walking or running is second nature and not given any thought or consideration. However, the movement of a person's legs can actually yield a wealth of information that can be used to assist the person. The study of a person's leg movement can reveal possible physiological or mental issues regarding the person. For instance, if a person has an injury, the person may favor one leg over the other; or if the person's cognitive capabilities start changing, the way the person walks may manifest the person's change in cognitive capabilities. Additionally, athletes or a typical person who desires to exercise could use the real time feedback of how their feet are striking the ground as they walk or jog or run to determine if they are walking or jogging or running properly with the way they want to walk or jog or run, and if not, to use the real time feedback to modify how they are walking or jogging or running as they desire.

Separately, a person's foot or feet can be used as input to a controller to cause changes to be implemented. For instance, anyone who drives already uses their feet or at least one foot to control the movement of a car by the way they press the gas pedal or the brake pedal. If more subtle movements of the foot or feet could be detected, then more refined inputs could be generated to provide a greater range of controls or directions, analogously to how the movement of fingers or hands can be used to operate a multitude of devices.

### BRIEF SUMMARY OF THE INVENTION

The present invention pertains to an apparatus for a shoe which provides information to a remote computing device having a user application. The apparatus comprises a first insert configured to be disposed in the shoe having a wireless transmitter, at least one sensor for sensing pressure that provides an antialiased image of pressure, and a controller in communication with the sensor and the transmitter which receives the pressure sensed by the sensor and transmits data associated with the pressure sensed through the transmitter to the device.

The present invention pertains to a method for providing information about a user to a remote computing device having a user application. The method comprises the steps of sensing pressure with at least one sensor of a first insert configured to be disposed in a first shoe of the user. The sensor provides an antialiased image of pressure. There is the step of providing data associated with the pressure sensed by the sensor to a controller of the first insert. There is the step of receiving data at a receiver of the first insert from a second insert configured to be disposed in a second shoe of the user which communicates data regarding pressure concerning the second insert with the first insert. There is the step of aggregating by the controller of the first insert the data of the first and second inserts. There is the step of transmitting the aggregated data to the device.

The present invention pertains to a novel pressure wireless pressure sensitive shoe insert that provides an antialiased image of pressure, which can target any desired region or regions of the foot to detect changes in pressure as well as to detect features such as phalanges, tarsals, metatarsals and the heal of the foot. The shoe inserts in shoes of a user communicate with each other to aggregate data, before sending said data to a users' computing device, such as a mobile phone or computer.

BRIEF DESCRIPTION OF THE SEVERAL VIEWS OF THE DRAWING

In the accompanying drawings, the preferred embodiment of the invention and preferred methods of practicing the invention are illustrated in which:

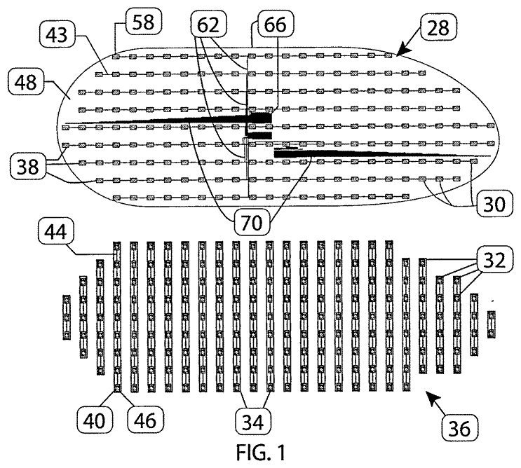

FIG. 1 shows Top and Bottom Layers of a left foot sensor, Where the top layer (pictured below) consists of vertical strips of conductor and FSR that are laid atop the bottom layer.

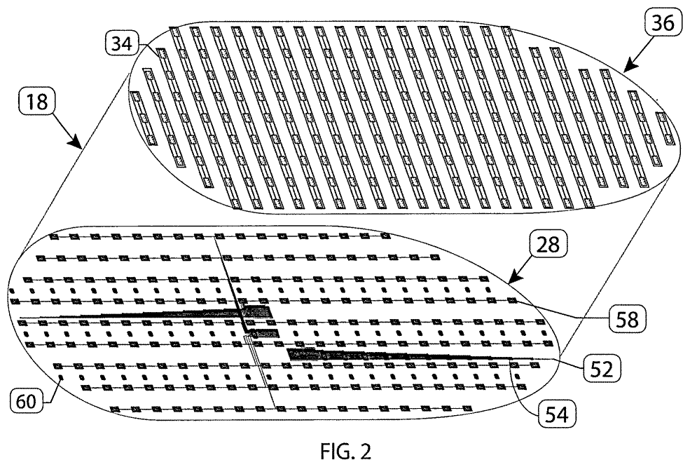

FIG. 2 shows Strips that form the top layer above the bottom layer.

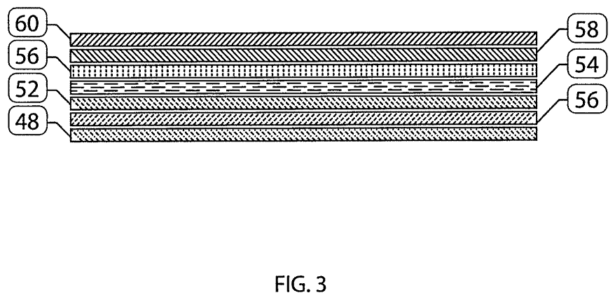

FIG. 3 shows the Print Order for Sensor Base—Bottom to Top: Substrate, Conductor (e.g., silver), Conductive adhesive, Dielectric, Silver, FSR, Adhesive.

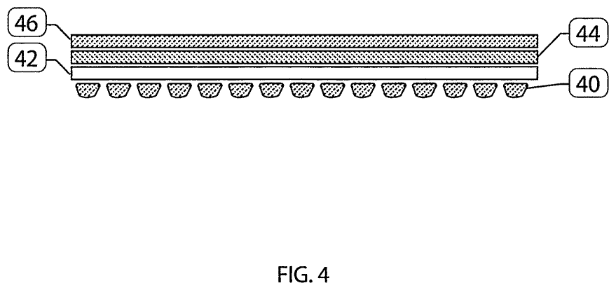

FIG. 4 shows the Print Order for Sensor Strips—Bottom to Top: Protrusion/Spacer, Substrate, Conductor (e.g., Silver or Copper), FSR.

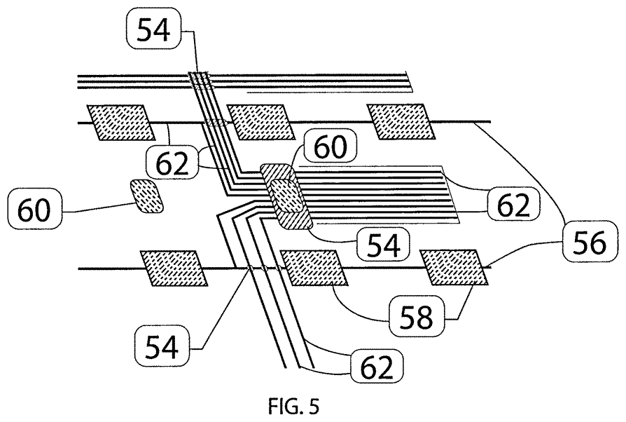

FIG. 5 shows an example of routing regarding the bottom sensor traces.

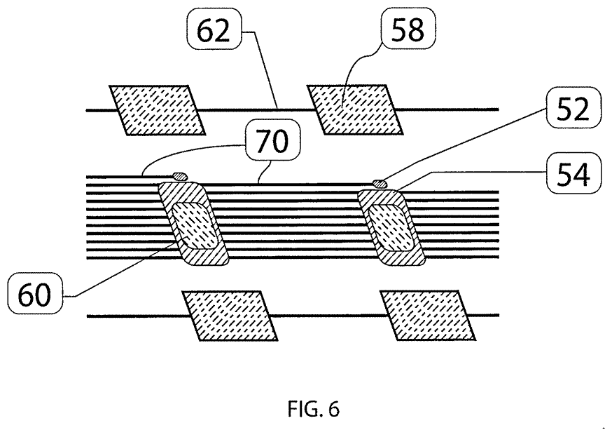

FIG. 6 shows Close up of Strips with Conductive adhesives, dielectric, and mundane adhesive.

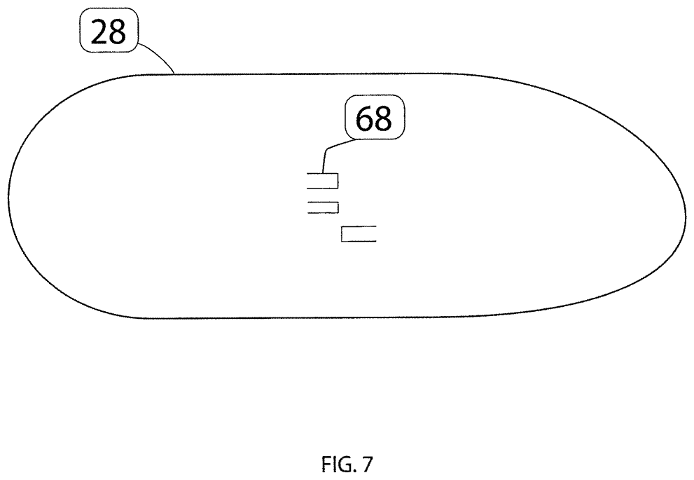

FIG. 7 shows FPC Cutouts.

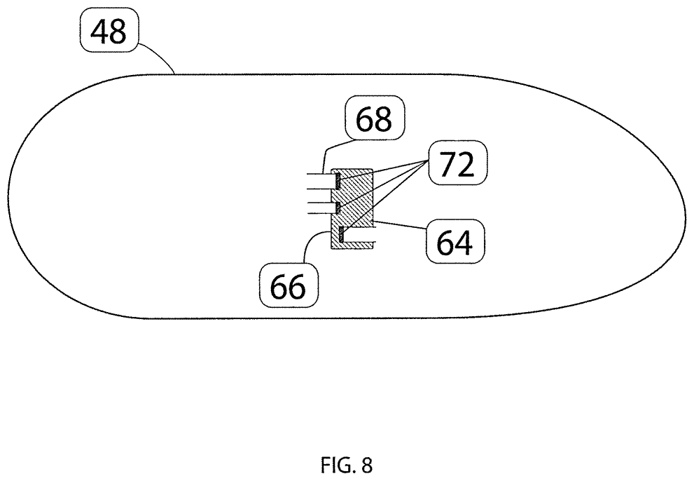

FIG. 8 shows Cut out Flat Printed Cables That plug into a PCB inset into an insole.

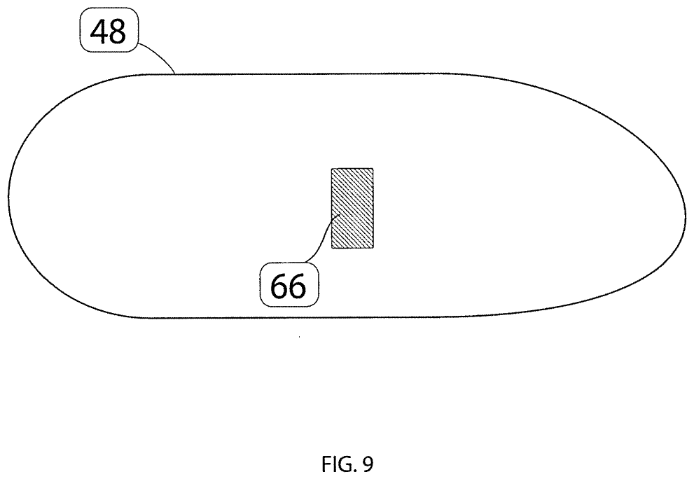

FIG. 9 shows a Shoe Insert with cutout for PCB.

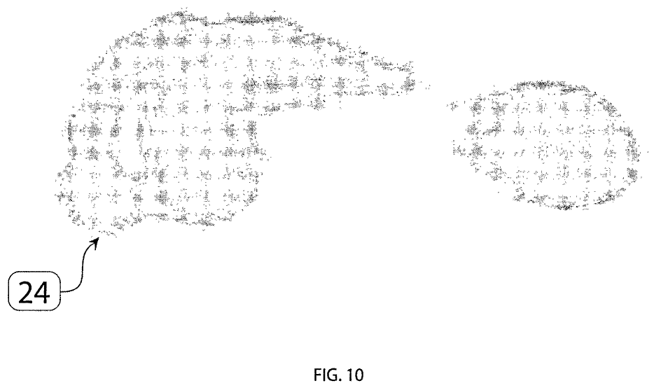

FIG. 10 shows a computer-generated image of an antialiased image.

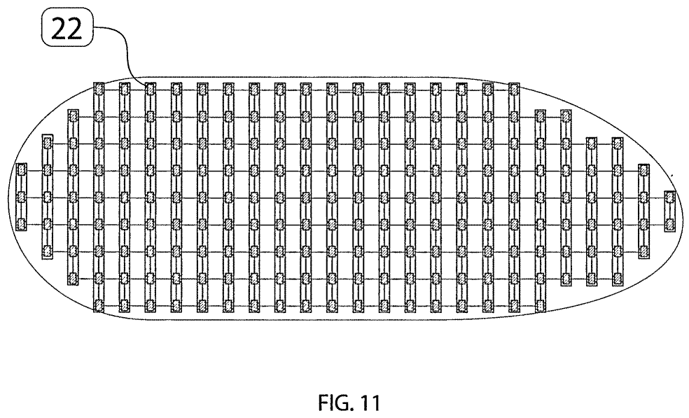

FIG. 11 shows the Left foot Top and Bottom Layer Sensor layout with ½ in spacing sensal layout between the sensors.

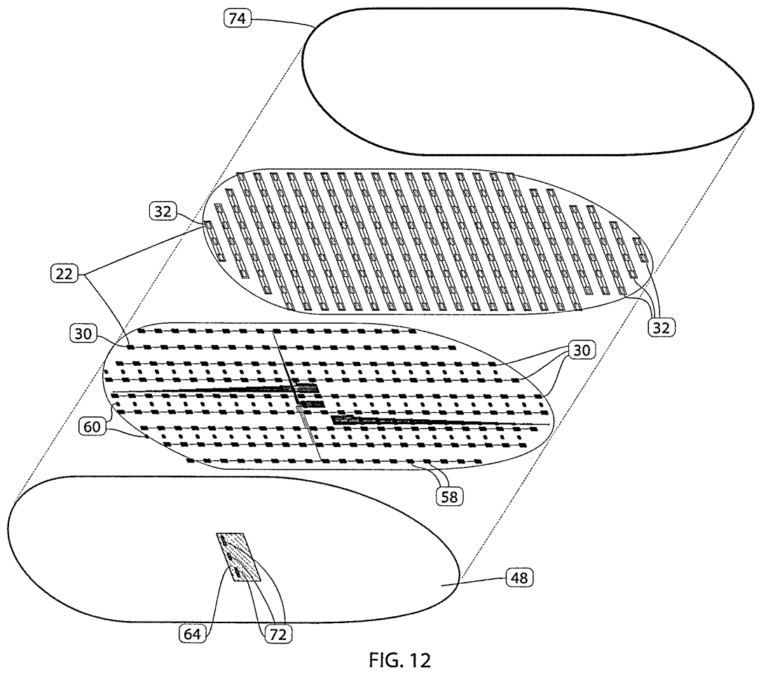

FIG. 12 is an Exploded View of Insert, PCB, sensor film, and top insert layer.

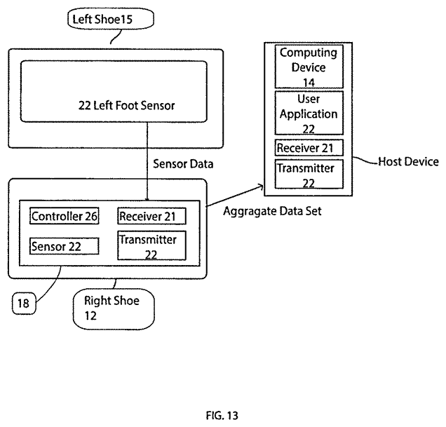

FIG. 13 is a block diagram of the apparatus of the present invention.

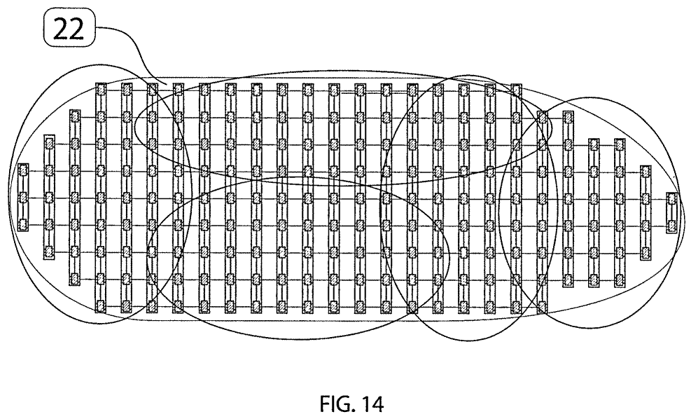

FIG. 14 shows Elliptical areas of interest in one embodiment.

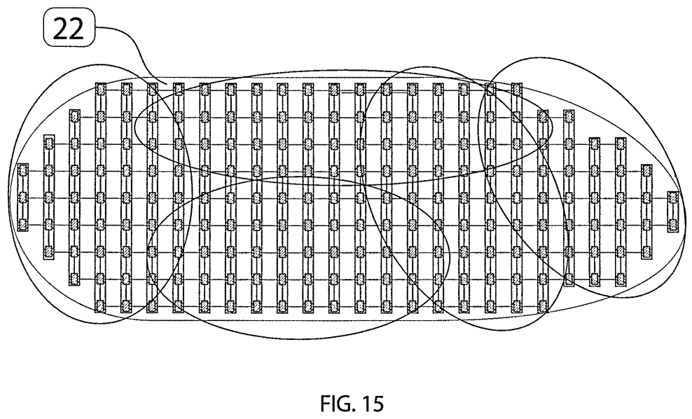

FIG. 15 shows Better fitting for ellipses.

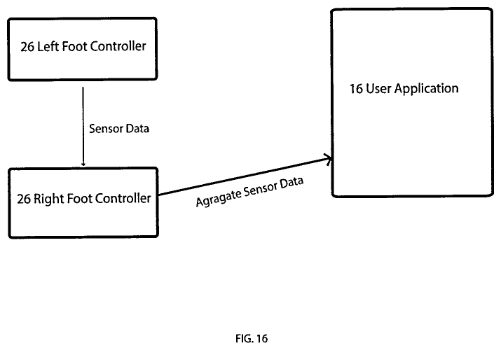

FIG. 16 is a Communication Diagram.

### DETAILED DESCRIPTION OF THE INVENTION

Referring now to the drawings wherein like reference numerals refer to similar or identical parts throughout the several views, and more specifically to FIG. 13 thereof, there is shown an apparatus 10 for a shoe 12 which provides information to a remote computing device 14 having a user application 16. The apparatus 10 comprises a first insert 18 configured to be disposed in the shoe 12 having a wireless transmitter 20, at least one sensor 22 for sensing pressure that provides an antialiased image 24 of pressure, as shown in FIG. 10, and a controller 26 in communication with the sensor 22 and the transmitter 20 which receives the pressure sensed by the sensor 22 and transmits data associated with the pressure sensed through the transmitter 20 to the device 14.

The apparatus 10 may include a second insert configured to be disposed in a second shoe 15 of the user which communicates data regarding pressure concerning the second insert with the first insert 18. The first insert 18 may have a receiver 21. The controller 26 of the first insert 18 aggregating the data of the first and second inserts before sending said aggregated data to the device 14.

The first insert 18 may include a bottom layer 28 having a plurality of sensor base portions 30 and a plurality of sensor top portions 32 disposed on the sensor base portions 30, as shown in FIGS. 1, 2 and 12. A sensor-base portion 30 with a sensor top portion 32 disposed on the sensor-base portion 30 forming a single sensor 22. The plurality of sensor top portions 32 may be arranged on strips 34 to form a top layer 36. The sensor-base portions may be arranged in rows 38 on the bottom layer 28 with the strips 34 positioned perpendicularly relative to the sensor base portions. As shown in FIG. 4, the top layer 36 may comprise protrusions 40, a top substrate 42 on top of and in contact with the protrusions 40, top conductors 44 on top of and in contact with the top substrate 42 and top FSR 46 on top of and in contact with the top conductors 44.

As shown in FIG. 3, the bottom layer 28 may comprise a bottom substrate 48, bottom conductors 43 on top of and in contact with the bottom substrate 48, conductive adhesive 52 on top of and in contact with the bottom conductors, dielectric 54 on top of and in contact with the conductive adhesive 52, silver 56 on top of and in contact with the dielectric 54, bottom FSR 58 on top of and in contact with the silver 56, and adhesive 60 on top of and in contact with the bottom FSR 58. The bottom layer 28 may have bottom sensor traces 62 that electrically connect the plurality of bottom sensor portions with the controller 26, and may have top sensor traces 70 that electrically connect the plurality of top sensor portions with the controller 26, as shown in FIGS. 5 and 6. The controller 26 may be part of a printed circuit board 64, as shown in FIGS. 6, 7, and 8. The bottom layer 28 having a cut out 66 in which the printed circuit board 64 is disposed. The cut out 66 with the printed circuit board 64 may be position under the arch or instep of the foot when the foot is in the shoe 12.

The bottom layer 28 may have printed cables 68 to which the bottom sensor traces 62 and top sensor traces 70 are connected and end. The cables 68 are plugged into connectors 72 on the printed circuit board 64. The apparatus 10 may include an elastomer layer 74 disposed atop the top layer 36, as shown in FIG. 12. The controller 26 may send a power signal out through the bottom layer 28 traces to the bottom sensor portions and when force is applied to the top sensor portions, the power signal continues through at least one of the top sensor portions through the top layer 36 traces and back to the controller 26. When the top sensor portions are moved under the applied force to contact the bottom sensor portions, resistance between the top sensor portions and the bottom sensor portions is reduced and voltage is increased.

The present invention pertains to a method for providing information about a user to a remote computing device 14 having a user application 16. The method comprises the steps of sensing pressure with at least one sensor 22 of a first insert 18 configured to be disposed in a first shoe 12 of the user. The sensor 22 provides an antialiased image 24 of pressure. There is the step of providing data associated with the pressure sensed by the sensor 22 to a controller 26 of the first insert 18. There is the step of receiving data at a receiver of the first insert 18 from a second insert configured to be disposed in a second shoe 12 of the user which communicates data regarding pressure concerning the second insert with the first insert 18. There is the step of aggregating by the controller 26 of the first insert 18 the data of the first and second inserts. There is the step of transmitting the aggregated data to the device 14.

The present invention pertains to a novel pressure wireless pressure sensitive shoe insert 18, as shown in FIG. 2, that provides an antialiased image 24 of pressure, which can target any desired region or regions of the foot to detect changes in pressure as well as to detect features such as phalanges, tarsals, metatarsals and the heel of the foot. The shoe inserts in shoes of a user communicate with each other to aggregate data, before sending the data to a users' computing device 14, such as a mobile phone or computer. The wireless pressure sensitive shoe insert 18 comprises a bottom layer 28 having a plurality of sensor base portions 30. The shoe insert comprises a plurality of sensor top portions 32 disposed on the sensor base portions 30. A sensor-base portion with a sensor top portion disposed on the sensor-based portion forming a single sensor 22. The plurality of sensor base portions 30 and the plurality of sensor top portions 32 disposed on the sensor-based portions forming a plurality of sensors that provides an antialiased image 24 of pressure, as shown in FIG. 12.

Capabilities:

1. Motion sensing
a. Walking start/stop
b. Running start/stop
c. Balance/center of gravity
d. Intensity level of activity

2. Command and control via Bluetooth
a. Toe tap
b. Toe curl
c. Heel tap
d. Foot slide (directional)
e. Foot press hard
f. Foot pronation or supination

Supposing that a user is wearing shoes with the inventive inserts that are connected to a smartphone app via Bluetooth Low Energy and the user is also wearing a Bluetooth headset, these are some use cases:

1. A user can control music/volume and other app features through foot gestures that can include:
a. Starting to run
b. Tapping toes/heels a certain number of times
c. Tapping toes/heels and then sliding foot in a certain direction
d. Control gestures can involve both feet. Each of the two feet can doing complementary gestures. For example, the user can set a mode with her left foot. One example of such a mode setting gesture is supinating the foot so that only the outermost edge of the foot is touching the floor. Performing that gesture that will activate a mode in which, for example, varying total weight on the right foot will modulate a parameter, while putting more pressure on the toe or on the heel of the right foot will modulate another one-dimensional parameter, while pronating or supinating the right foot will modulate yet another parameter. This is merely one example of many such possible combinations of (1) setting a mode by positioning one foot in a particular way while then (2) using the other foot to vary parameters within the multidimensional parameter space associated with the mode which has been specified by the first foot. These values are understood by the software as dimensions of parametric control of some application that may be running on the app of the SmartPhone that is in communication with the feet sensors.
e. When using a one-foot gesture to enter a particular mode—as in the above example where the left foot is supinated—the other foot can perform discrete gestures, such as tapping one or more times with the toe, with the heel or with the outer edge of the foot or with the foot flat on the ground, or with any sequence of such tapping movements. Because such tapping gestures can be seen as a method of entering discrete values in base N (where N=4 in the case of heel/toe/outer-edge/flat-on-ground taps), arbitrarily large number of possible tapping combinations is available through this method. Therefore, this input method can be used as a form of coded entry for text or other digital information. One such possible encoding is Morse code, which can, for example, be effected by a kinematic gesture such as tapping the right toe for varying lengths of time while supinating the left foot, or, alternatively, by a dynamic (that is, varying force only, with no accompanying visible movement), of varying the weight between the toe and the heel of the right foot, while supinating the left foot.
f. Such modes can alternatively be specified by a gesture that involves both feet. For example, the user can simultaneously supinate both feet, so that both feet are supported on their outermost edge. While in this mode, the user can shift her weight, varying the relative body weight that is over the left foot or the right foot. This weight shifting is then understood by the software as a one-dimensional control parameter, which can be transmitted to an app which is running on the user's SmartPhone.

2. If a user loses his balance during a high intensity workout, a tone can play on the user's headset to alert the user of possible risk of injury
a. The pitch of the tone could vary based on the intensity and direction of the loss of balance
b. This could potentially be used to help the user improve balance during high intensity workouts
c. For more at-risk users, a low-level tone during normal walking could be used to provide feedback as to best practices during walking, to maximize balance and minimize the probability of falls.

Printing Techniques (incorporated by reference, herein):

https://basiccopper.com/6-x-4-1-rolled-sh.html?gclid=EAMIQobChMI8ofbwJu_2wIVSGB-Ch1V0AmTEAQYASABEgJ-wvD_BwE

PHASER 8560N COLOR SOLID INK PRINTER for $843.65 (Jan. 16, 2009)

Xerox Phaser 6510 solid ink printer for $220 from Staples

Instructional video on printing PCB's with solid ink printer: https://www.youtube.com/watch?v=s049jNdAnPE

Instructables of printing flex circuits

Kapton Foil for printing traces

Pyralux Film for Printing flex circuits

Dupont 7802 resistor paste (FSR)

U.S. patent application Ser. No. 15/362,438; U.S. Pat. Nos. 9,158,369 and 9,524,020, all of which are also incorporate by reference herein.

Components:

Parts

Sensor film

Substrate (generally a PET), conductive material for signal traces, dielectric 54, pressure variable resistance material (FSR) adhesive

Control Electronics:

Add BOM(IC, Mux, switches, Bluetooth LE module, zif's,)

One embodiment BOM for controller.

| | | | Manufacturer |
|---|---|---|---|
| Comment | Description | Manufacturer 1 | Part Number 1 |
| Cap | Capacitor | | C1005X7R1H104K |
| Cap | Capacitor | | CGJ2B2C0G1H101J |
| Cap | Capacitor | | C1005X5R0J475M |
| Cap Pol3 | Polarized Capacitor | AVX | TCJA106M010R0300 |
| | (Surface Mount) | | |
| Pot | 74HC4051 quad pot, | Microchip | MCP4362- |
| | 14-Pin TSSOP, | Technology | 103E/ST |
| | Extended Temperature | | |
| Ferrite Bead | Inductor | Bourns | MU1005-600Y |
| COM0 | Buffers & Line | TI | SN65HVD33DR |
| | Drivers 3 V Full-Dplx | | |
| | Driver and Receiver | | |
| LED02 | Typical RED GaAs | Avago | HSMC-C170 |
| | LED | Technologies | |
| Mux | Multiplexer Switch | Analog | ADG706BRUZ |
| | ICs 16:1 25 MHz 2.5 Ohm | Devices | |
| | CMOS | | |
| FH28-50S- | | Hirose | FH28-50S- |
| 0.5SH(05) | | | 0.5SH(05) |
| Res | Resistor | Vishay | CRCW04021K00FKEDHP |
| Res | Resistor | Vishay | CRCW040210K0JNEDHP |
| Res | Resistor | Vishay | CRCW040210R0FKEDHP |
| Res | Resistor | Vishay | CRCW0402270RFKED |
| SPDT | FSA2267 Low- | | FSA2267AL10X |
| | Voltage, Dual-SPDT | | |
| Conn 1 | 9 pin PFC Connector | Molex | 0527450997 |
| PIC24HJ256GP610- | High Speed General | Microchip | PIC24HJ256GP610- |
| I/PT | Purpose 16-Bit Flash | Technology | I/PT |
| | Microcontroller, | | |
| | 256 KB Flash, 16 KB | | |
| | RAM, 100-Pin TQFP, | | |
| | Industrial | | |
| | Temperature, Tape | | |
| | and Reel | | |
| TS912AIDT | Operational | STMicroelectronics | TS912AIDT |
| | Amplifiers - Op Amps | | |
| | Dual Rail-to-Rail 3 V | | |
| Volt Reg | Voltage Regulator | Infineon | IFX25001MEV33 |
| Bluetooth LE | Bluetooth LE Module | Microchip | RN4020- |
| Module | | Technology | V/RMBEC133 |
| | | | Part Count |

This invention consists of a set of durable sensors 22 embedded in a user's footwear and a control device 14 such as a mobile device or computer.

The production sensor 22: in one embodiment, the sensors are each a pressure sensing matrix consisting of a plurality of variably resistive nodes, as described in U.S. Pat. No. 9,411,457. Each sensor 22 consists of one base piece and that has the size and shape of a shoe insert and several strips 34 which are adhered laterally across the sensor 22 regular intervals aligning with the matrix pattern of the base piece as in FIGS. 1 and 2, each with several layers of functional inks. Printed layers of the sensor 22 consist of the following: conductor for signal traces, a force sensitive resistor (FSR) ink, dielectric 54, conductive adhesives, and a physical spacer ink where print ordering may be as shown in FIGS. 3 and 4.

The routing of current happens on the base piece of the sensor 22, utilizing conductive inks and dielectric 54 to create multiple layers of signal traces as in FIG. 5, where conductors cross, dielectric 54 is printed after the first conductor is laid down, then the second conductor is printed, where the dielectric 54 then prevents a short signal. Conductive signal traces run the length of the sensor 22 and at intervals strips 34 consisting of a conductor across the length of the strip, FSR ink at intervals corresponding to the FSR on the base piece, a spacer printed on the opposite side of the strip situated over the FSR are adhered to the base. A conductive adhesive 52 is used to carry current across these strips 34, and a mundane adhesive 60 is used in order to firmly secure the strip to the base as in FIG. 6, where the conductive adhesive 52 provides secure conductive connection, the dielectric 54 prevents shorts.

The signal trace end at a flat printed cable (FPC) either at the heel or at the instep of the first insert 18, which plugs into a ZIF or LIF connector on a printed circuit board 64 (PCB) which contains the control electronics for powering and reading the sensor 22 and also analyzed the data and transmits the results to the control device 14 (mobile phone, tablet, laptop computer, etc.) In the embodiment described herein, the FPC's are located approximately at the instep. The FPC are cut free from the substrate on three sides via die cut, laser cut, or other standard method resulting in three FPC's still connected at one edge to the sensor 22 to carry signal to the PCB, as shown in FIG. 7. The FPC's then plug into a printed circuit board 64 via FPC connectors such a zero input force (ZIF) or low input force (LIF) connector as shown in FIG. 8. The PCB is embedded within a shoe insert (FIG. 9 shows the cutout 66 in the bottom substrate 48 for the PCB). The sensor 22 is then plugged into the PCB and adhered onto the insert (FIG. 12). A layer 74 of soft, flexible elastomer like material such as Rubber, PVC, TPE, cut into the shape of the insert, is then placed over the sensor 22 (FIG. 12).

The sensor 22 information for each foot is generated in the same manner as in U.S. Pat. No. 9,411,457. One of the two PCBs act as host and the other client, where the client sends the information generated by the sensors 22 in the second insert of the left shoe 15 to the host via a wireless communication protocol such as Bluetooth, which is used in this embodiment. In this embodiment, the right shoe 12 PCB acts as host and the left shoe 15 PCB as client. The Host then acts as a client to send both sensor data sets wirelessly (in this embodiment this is done with Bluetooth as well) to another device 14 such as a mobile phone or computer (FIG. 13).

Testing sensors 22: Sensors 22 can also be designed that will act as a testing rig, which will allow the optimal configuration of the sensors 22 to be determined. Start with a base piece having sensors densely packed (¼″) in a regular grid formation. Then prototype the strips 34 in a manner that allows various configurations to be tested, as well as spacings, and resistance levels. Next, screen print onto a substrate such as MELINEX® ST505 conductive lines (or start with a conductive material [e.g., copper foil]), then screen print the FSR ink in strips 34 spaced such that they correspond to the sensor base portions 30 on the bottom layer 28 of the sensors 22 at any interval chosen. For example, the strips 34 could have an FSR spacing of ¾″, connecting to every third sensor base portion 30 on the bottom layer 28. On the back side of the substrate, laser cut strips 34 of FR4 are adhered at 2 mil (approx. 0.5 mm) over the FSR. The FR4 then can act as a spacer or protrusion on the sensor 22 when assembled. Then laser cut strips 34 from the substrate perpendicular to the FSR+FR4 strips 34, which can then be adhered to the bottom layer 28 of the sensors 22 perpendicular the columnal traces on the bottom layer 28. In this manner, varying resolutions are able to be tested (including different resolutions in different areas of the sensors 22). This also enables various pressure ranges to be tested by modifying the FSR ink before the printing process. Again, even different pressure ranges can be tested for different parts of the sensors 22.

This, altogether, allows one to determine the optimal specifications of sensors 22 for different applications quite quickly (i.e., in days as opposed to several weeks and up to two months in a traditional manufacturing process). Scanning or reading values from the sensors 22 is done in the same method as in U.S. Pat. No. 9,411,457.

Each apparatus ideally consists of at least two sensors and two printed circuit board control units (MCU based control units [i.e., controllers]). Each sensor 22 will be connected to a controller 26 via flat printed cables 68 plugged into a ZIF or LIF connector. The sensors are designed for each foot (a left and right version) and embedded into a shoe insert. The sensors are less than ⅓ mm thick (depending on the substrates into which they are printed) and the controllers are less than 4 mm (again, depending on the PCB substrate thickness and the thickness of components used). In this embodiment, the sensor film is ⅓ mm thick and the controller 26 is 4 mm thick. The sensors 22 and controller 26 will be embedded within the arch portion of the insole. FIG. 11 shows the sensor top portions 32 aligned over the sensor base portions 30 to form the sensors 22, so there is a one to one correspondence of one sensor top portion 32 disposed over one sensor base portion 30.

The controller 26, when activated, will gather time varying frames of pressures applied to the sensor 22 surface. The controller 26 will then compile that pressure information into a small number of bytes representing a paradigm of the foot's pressure-based command sequence(s)) or a compressed version of the complete foot pressure image. For each frame that a sensor 22 is scanned, there will be a corresponding value for each sensor. In this embodiment, using a 12-bit ADC onboard the MCU, the value is 0-2\hat{ }12 (0-4096) for each of the 201 sensors.

Since there will often be a number of sensors 22 with a value of zero, one useful method of compression is run-length encoding, where all zeros are represented by a single number to indicate the number of zeros that follow (e.g., −31 indicates 31 zeros replace the −31). In order to reduce communication packet size, processing that can be done onboard the MCU can represent a set of values representing targeted regions of the sensors 22. For example, one area around the end of the sensors 22 or where a user's toes might be (phalangeal area), one area for the balls of the feet (metatarsal area), the outside of the foot (tarsal), the instep (or arch), and the area of the sensors 22 that might cover the heel (below the calcaneus). These regions may overlap, but the data for each sensor 22 is added to a weighted sum to determine total force for each region and the total center of pressure. In the embodiment described above, this would result in five regions each represented by three values: xCOP, yCOP, and total relative force. The result is a data packet that consists of 15 data values for each foot, or 30 data values total, as opposed to a worst case of 402 data values for both feet. This has the advantage of smaller data packets (reduced power consumption), reduced communication complexity (data payload in Bluetooth is generally limited to 251 bytes, thus 402 data values would necessitate a single frame to be transmitted over multiple packets), and a fast communication rate, as the max packet size is considerably smaller. The information from one foot insert sensor (the slave or client) will be sent to the other foot insert sensor (the master or server) via a communication protocol such as Bluetooth. This embodiment is implemented via Bluetooth LE. The master PCB then communicates to a separate device 14 such as a mobile phone, tablet, computer or other device via a communication protocol such as Bluetooth LE. Again, the preferred implementation uses Bluetooth LE.

Algorithm for computing the aggregate value for each elliptical region (FIGS. 14 and 15):

The weighted sums are calculated as follows: For each ellipse shaped region for which the PCB is calculating [xCOP, yCOP, force], 6 coefficients [ax,bx,cx,ay,by,cy] are pre-stored in a table which resides in memory on the microprocessor of the PCB. These coefficients are used to transform any given sensel location [x,y] to a transformed location [x′, y′]=[ax * x+bx*y+cx, ay*x+ay*y+cy]. This transformation function for ellipse n can be denoted as Tn([x,y]).

The transformed location [x′,y′] is then used to compute a radially symmetric kernel function, which has its greatest value at the origin and drops off to zero at a radius of 1.0. In one embodiment, this kernel is defined as: (1−sin(2*π*min(1, x′*x′+y′*y′))/2. This kernel function can be denoted as K([x′,y′]).

The sensors 22 of an insert are scanned, and for each sensel location [x,y], with force f, values X,Y,F are accumulated as follows:

First, for every ellipse n, initialize Xn=0 and Yn=0 and Fn=0.

Then for each sensel [x,y)]:

For each ellipse n:

Let f=K(Tn([x,y]))

Xn+=f*x

Yn+=f*y

Fn+=f

The values transmitted for each ellipse are then: [xCOP, yCOP, force]=[Xn/Fn, Yn/Fn, Fn].

Sending the aggregate data from the insert's on-board microprocessor to a host device 14:

The aggregate data from both shoe insert sensors is then interpreted by the receiving device 14 and the software application (the app) on the device 14 such that it can then provide feedback to the user or other interested parties. For any particular application or use of this system, the ‘app’ can then analyze the resulting data to provide a response to the user or other interested parties. In one case, the application can look at the change in relative positions of the phalanges region vs the metatarsal region to indicate if a user is curling her toes to better grip the ground, which may indicate a balance issue. The ‘app’ can provide this information to the user.

Use Case

One use case scenario is to use this together with a head worn audio device 14, which is also in wired or wireless communication with the app that is running on the phone. In one user scenario, the movements of the user's footsteps are analyzed by the phone app and sent as audio feedback to the head worn audio device 14. For example, if the balance of total weight distribution between the left and right feet can be delivered by the phone app to the head worn audio device 14 as an audible tone whose apparent left/right position corresponds to the left/right weight distribution of the force imparted to the ground by the left and right foot, respectively.

The above use case can be implemented as follows: The total weight of the left foot in any given time interval of measurement, (for example, two seconds) is sent to the phone app as value L, and also the total weight of the left foot in the same time interval of measurement is sent to the phone app as value R.

The audio volume of the audio tone sent by the phone app to the user's left ear is proportional to L, whereas the audio volume of the audio tone sent by the phone app to the user's right ear is proportional to R.

To the user, the sensation will be that the apparent left/right spatial location of the audio tone will indicate the relative weight that the user is placing on each foot while standing or walking.

This application can be used to enable patients recovering from injuries to monitor their own walking to improve their health and balance. For example, often when people experience a leg or foot injury, they “favor” one foot by placing more weight on the other foot, rather than placing weight equally, which is healthier for recovery from injury. Being able to “hear” the undesired weight imbalance can help the recovering patient to walk and stand in a more balanced and therefore healthy manner.

Use Case—Team Activities

Another use case scenario that combines the use of a head worn audio device 14 and an app with sensors 22 is team activities, such as professional or amateur sports. In this use case, each time a team member (a “player”) takes a step, that player's insert with sensors 22 will send a wireless message to an app which is tracking the footsteps of all of the members of the team.

By measuring the duration and intensity of each footstep combined with the intensity of the bluetooth signal of each Tactonic sensor (using an existing bluetooth LE beacon protocol), the app can track the relative position of all players as well as their direction of travel.

As players move around the playing field, their footsteps can thereby trigger the app to generate an audible tone that is sent in real time to other players on the team.

Using the app, an administrator will be able to assign a personalized tone to a specific player or to a group of players. In this way players on the team will be able to identify either the unique group or unique person to which the tone belongs.

These tones will be recreated in a 3D sound field inside of each player's headset so that each player can observe a realtime audio “map” of his or her other team members. For example, if one of the other team members is moving closer to a player, that player's tone will appear proportionally louder with each step. This variation in loudness can, in one embodiment, simply be a linear function of 1/distance. Other information can be conveyed by this audible tone as well. For example, if the other team member is moving with heavy steps, the tone can be made shorter in duration to indicate this variation.

This protocol can be useful for instances of team practice where each player has full line-of-sight of other team members but will also in instances where line-of-sight visibility between players is limited, which is often the case in real-life situations.

Some examples of how audio cues from this technology can provide greater situational awareness include:
Team sports such as football
Players on the offensive line will be able to judge whether a quarterback is scrambling to avoid defensive linemen
   The quarterback will be able to judge whether a receiver is blocked by defenders or has broken free
Life or death situations such as emergency first responders entering a building
   Will be able to judge whether other members of the first responder team are moving in formation or are stopped

Only a very small low power logic on the
insert is needed, which can easily fit in a
low profile board within the arch of the
foot.
Regenerative power from walking foot
movement can be used to recharge [Hsu]
incorporated by referene herein.
Force statistics for a foot are computed in
this board, producing 3 values: (a) total
weight, (b) pronate/supinate distribution,
(c) heel/toe distribution, all of which can
be sent within a 4 byte data word per time
slice. This allows communication with
the phone app to be via low bandwidth
bursts using a low power protocol such as
802.15.4.

References, all of which are incorporated by reference, herein:

Hsu, T.-H., Manakasettharn, S., Taylor, J. A., & Krupenkin, T. (2015). Bubbler: A Novel Ultra-High Power Density Energy Harvesting Method Based on Reverse Electrowetting. Scientific Reports, 5, 16537. http://doi.org/10.1038/srep16537

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

1. An apparatus for a shoe which provides information to a remote computing device having a user application comprising:
a first insert configured to be disposed in the shoe having a wireless transmitter, at least one sensor for sensing pressure that provides an antialiased image of pressure, and a controller in communication with the sensor and the transmitter which receives the pressure sensed by the sensor and transmits data associated with the pressure sensed through the transmitter to the device.

2. The apparatus of claim 1 including a second insert configured to be disposed in a second shoe of the user which communicates data regarding pressure concerning the second insert with the first insert, the first insert having a receiver, the controller of the first insert aggregating the data of the first and second inserts before sending said aggregated data to the device.

3. The apparatus of claim 2 wherein the first insert includes a bottom layer having a plurality of sensor-base portions and a plurality of sensor top portions disposed on the sensor-base portions, a sensor-base portion with a sensor top portion disposed on the sensor-base portion forming a single sensor.

4. The apparatus of claim 3 wherein the plurality of sensor top portions arranged on strips to form a top layer.

5. The apparatus of claim 4 wherein the sensor-based portions arranged in rows on the bottom layer with the strips positioned perpendicularly relative to the sensor base portions.

6. The apparatus of claim 5 wherein the top layer comprises protrusions, a top substrate on top of and in contact with the protrusions, top conductors on top of and in contact with the top substrate and top FSR on top of and in contact with the top conductors.

7. The apparatus of claim 6 wherein the bottom layer comprises a bottom substrate, bottom conductors on top of and in contact with the bottom substrate, conductive adhesive on top of and in contact with the bottom conductors, dielectric on top of and in contact with the conductive adhesive, silver on top of and in contact with the dielectric, bottom FSR on top of and in contact with the silver, and adhesive on top of and in contact with the bottom FSR.

8. The apparatus of claim 7 wherein the bottom layer has bottom sensor traces that electrically connect the plurality of bottom sensor portions with the controller, and connect the plurality of top sensor portions with the controller.

9. The apparatus of claim 8 wherein the controller is part of a printed circuit board, the bottom layer having a cut out in which the printed circuit board is disposed.

10. The apparatus of claim 9 wherein the bottom layer has printed cables to which the bottom sensor traces and top sensor traces are connected, the cables are plugged into connectors on the printed circuit board.

11. The apparatus of claim 10 including an elastomer layer disposed atop the top layer.

12. The apparatus of claim 11 wherein the controller sends a power signal out through the bottom layer traces to the bottom sensor portions and when force is applied to the top sensor portions, the power signal continues through at least one of the top sensor portions through the top layer traces and back to the controller, when the top sensor portions are moved under the applied force to contact the bottom sensor portions resistance between the top sensor portions and the bottom sensor portions is reduced and voltage is increased.

13. A method for providing information about a user to a remote computing device having a user application comprising the steps of:
sensing pressure with at least one sensor of a first insert configured to be disposed in a first shoe of the user, the sensor provides an antialiased image of pressure;
providing data associated with the pressure sensed by the sensor to a controller of the first insert;
receiving data at a receiver of the first insert from a second insert configured to be disposed in a second shoe of the user which communicates data regarding pressure concerning the second insert with the first insert;
aggregating by the controller of the first insert the data of the first and second inserts; and
transmitting the aggregated data to the device.

14. A wireless pressure sensitive shoe insert comprising:
a bottom layer having a plurality of sensor-base portions; and a plurality of sensor top portions disposed on the sensor-base portions, a sensor-base portion with a sensor top portion disposed on the sensor-based portion forming a single sensor, the plurality of sensor-base portions and the plurality of sensor top portions disposed on the sensor-based portions forming a plurality of sensors that provides an antialiased image of pressure.
